(19) United States
(12) Patent Application Publication
(10) Pub. No.: US 2007/0005130 A1
(43) Pub. Date: Jan. 4, 2007

(54) Biodegradable polymer for coating

(76) Inventors: Thierry Glauser, Redwood City, CA (US); Syed Faiyaz Ahmed Hossainy, Fremont, CA (US)

Correspondence Address: SQUIRE, SANDERS & DEMPSEY LLP, 1 MARITIME PLAZA, SUITE 300, SAN FRANCISCO, CA, 94111, US

(21) Appl. No.: 11/171,111

(22) Filed: Jun. 29, 2005

(51) Int. Cl. A61F 2/82; A61K 47/12

(52) U.S. Cl. 623/1.38; 623/1.42; 424/426

(57) ABSTRACT

Copolymer that includes units derived from malolactonate or malolactonic acid and coatings or medical devices formed thereof are provided.

### BACKGROUND OF THE INVENTION

1. Field of the Invention

This invention generally relates to implantable devices, such as stents or coatings on stents, formed of a material that contains malolactonate derived repeating units.

2. Description of the Background

Although stents work well mechanically, the chronic issues of restenosis and, to a lesser extent, stent thrombosis remain. Pharmacological therapy in the form of a drug-delivery stent appears a feasible means to tackle these issues. Polymeric coatings placed onto the stent serve to act both as a drug reservoir and means to control the release of a drug. Examples of the commercially available polymer coated products are stents manufactured by Boston Scientific. For example, U.S. Pat. Nos. 5,869,127; 6,099,563; 6,179,817; and 6,197,051, assigned to Boston Scientific Corporation, describe various compositions for coating medical devices. These compositions, which may optionally include a bioactive agent, provide to stents described therein an enhanced biocompatibility. U.S. Pat. No. 6,231,590 to Scimed Life Systems, Inc., describes a coating composition, which includes a bioactive agent, a collagenous material, or a collagenous coating optionally containing or coated with other bioactive agents.

A current paradigm in the art of biomaterials is the control of protein adsorption on the implant surface. Uncontrolled protein adsorption, which leads to mixed layer of partially denatured proteins, is a hallmark of a surface formed of current biomaterials when implanted. Such a surface presents different cell binding sites from adsorbed plasma proteins such as fibrogen and immunogloblulin G. Platelets and inflammatory cells such as monocyte/macrophages and neutrophils adhere to these surfaces.

Another limitation of current drug-delivery stents stems from the fact that the stent is a foreign body. Use of drug-delivery stents has proved successful by use of controlled release of anti-proliferative or anti-inflammatory drugs to control restenosis. However, drug-delivery stents still have a small, but measurable, incidence of sub-acute thrombosis. In addition, drug-delivery stents have not driven restenosis to zero levels, especially in more challenging patient subsets such as diabetics or patients with small vessels, and/or long, diffuse lesions. The present invention provides a polymeric material for coating implantable devices or forming an absorbable device such as a stent.

### SUMMARY OF THE INVENTION

Provided herein is a polymer derived from malolactonate or malolactic acid and another biocompatible molecule such as lactic acid or lactide. The polymer defined herein can be used alone or in combination with another biocompatible polymer and/or a biobeneficial material to form coatings on implantable medical devices or to form the implantable medical devices themselves.

The copolymer described herein can be made to contain basic or acidic pendant groups such as carboxylic acid or amino groups. Therefore, in some embodiments, the copolymer described herein can be used for (1) modulation of release rate of a drug by controlling the equilibrium uptake of water and (2) modulation of absorption rate by controlling the water uptake and absorption product transport through a coating containing the polymer. In addition, water is a plasticizing material and thus, higher water uptake can lead to improved coating integrity in a coating containing the copolymer described herein.

In some other embodiments, the polymer defined herein can be used for modulation of biological property of a coating. For example, the contact angle on a coating containing the copolymer described herein can be varied by changing the content of the polymer in the coating, leading to the modification of the biocompatibility of the coating. In addition, as described above, the polymer can be made to contain acidic or basic groups such as carboxylic acid or amino groups. Therefore, in some embodiments, these groups can be used for conjugation of biobeneficial moieties to the polymer.

In some embodiments, the copolymer described herein can be used alone or in combination with another biocompatible polymer (e.g., poly(D,L-lactic acid)), optionally with a biobeneficial material (described below) and/or one or more bioactive agents, for forming a coating on an implantable device (e.g., a stent) or for forming a fully absorbable device (e.g., a stent). Some exemplary bioactive agents are paclitaxel, docetaxel, estradiol, nitric oxide donors, super oxide dismutases, super oxide dismutases mimics, 4-amino-2,2,6,6-tetramethylpiperidine-1-oxyl (4-amino-TEMPO), tacrolimus, dexamethasone, rapamycin, rapamycin derivatives, 40-O-(2-hydroxy)ethyl-rapamycin (everolimus), 40-O-(3-hydroxy)propyl-rapamycin, 40-O-[2-(2-hydroxy)ethoxy]ethyl-rapamycin, and 40-O-tetrazole-rapamycin, 40-epi-(N1-tetrazolyl)-rapamycin (ABT-578), clobetasol, pimecrolimus, imatinib mesylate, midostaurin, prodrugs thereof, co-drugs thereof, and combinations thereof. The implantable device can be implanted in a patient to treat or prevent a disorder such as atherosclerosis, thrombosis, restenosis, hemorrhage, vascular dissection or perforation, vascular aneurysm, vulnerable plaque, chronic total occlusion, claudicationanastomotic proliferation for vein and artificial grafts, bile duct obstruction, ureter obstruction, tumor obstruction, or combinations thereof.

### DETAILED DESCRIPTION

Provided herein is a copolymer derived from malolactonate or malolactic acid and another biocompatible molecule such as lactic acid or lactide. The polymer defined herein can be used alone or in combination with another biocompatible polymer and/or a biobeneficial material to form coatings on implantable medical devices or to form bioabsorbable implantable medical devices. The term bioabsorbable encompasses both bioerodable and biodegradable.

The copolymer described herein can be made to contain basic or acidic pendant groups such as carboxylic acid or amino groups. In some embodiments, the copolymer described herein can be used for (1) modulation of release rate of a drug by controlling the equilibrium uptake of water and (2) modulation of absorption rate by controlling the water uptake and absorption product transport through a coating containing the polymer. In addition, because water is a plasticizing material, the copolymer described herein can be used to increase water uptake, leading to improved coating integrity.

In some other embodiments, the polymer defined herein can be used for modulation of biological property of a coating, e.g., for tuning of hydrophilicity/hydrophobicity of a coating or tethering of bioactive agent such as a peptide (e.g., RGD, CNP) or a drug to a coating. For example, the contact angle on a coating containing the copolymer described herein can be varied by changing the content of the polymer in the coating, leading to the modification of the biocompatibility of the coating. Contact angle can be indicative of the non-fouling property of a coating—the lower the contact angle, the more hydrophilic the coating. In addition, because the polymer can be made to contain acidic or basic groups such as carboxylic acid or amino groups, these groups can be used in some embodiments for conjugation of biobeneficial moieties to the polymer.

In some embodiments, the polymer described herein can be used alone or in combination with another biocompatible polymer (e.g., poly(D,L-lactic acid)), described below, optionally with a biobeneficial material (described below) and/or one or more bioactive agents, for forming a coating on an implantable device (e.g., a stent) or for forming a device itself (e.g., a stent). Some exemplary bioactive agents are paclitaxel, docetaxel, estradiol, nitric oxide donors, super oxide dismutases, super oxide dismutases mimics, 4-amino-2,2,6,6-tetramethylpiperidine-1-oxyl (4-amino-TEMP), tacrolimus, dexamethasone, rapamycin, rapamycin derivatives, 40-O-(2-hydroxy)ethyl-rapamycin (everolimus), 40-O-(3-hydroxy)propyl-rapamycin, 40-O-[2-(2-hydroxy)ethoxy]ethyl-rapamycin, and 40-O-tetrazole-rapamycin, 40-epi-(N1-tetrazolyl)-rapamycin (ABT-578), pimecrolimus, imatinib mesylate, midostaurin, clobetasol, prodrugs thereof, co-drugs thereof, and combinations thereof. The implantable device can be implanted in a patient to treat or prevent a disorder such as atherosclerosis, thrombosis, restenosis, hemorrhage, vascular dissection or perforation, vascular aneurysm, vulnerable plaque, chronic total occlusion, claudication, anastomotic proliferation for vein and artificial grafts, bile duct obstruction, ureter obstruction, tumor obstruction, or combinations thereof.

Polymers derived from malolactonate and lactic acid The polymer provided herein includes a moiety (A) derived from malolactonate or malolactonic acid and another biocompatible moiety (B) derived from another biocompatible material. The polymer can be a random copolymer or a block copolymer having Aₙ and Bₘ repeating untis that can be arranged in the form of AₙBₘ, AₙBₘAₙ, or BₘAₙBₘ, where n, n′, m and m′ are independent positive integers ranging from 1 to 100,000, e.g., about 1, 10, 20, 50, 100, 200, 500, 1,000, 2,000, 5,000, 10,000, 20,000, 50,000, or 100,000. In some embodiments, the polymer can be statistical copolymer, alternating copolymer or periodic copolymer as is understood by one of ordinary skill in the art. In some further embodiments, the polymer can include one or more moieties or blocks so as to form an ABC or ABCD type copolymer.

In some embodiments, the copolymer described herein contains repeating units of the following structure:

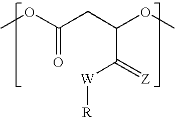

wherein Z is O, S or NR¹,

wherein W is absence or O, S, or NR², and

where R, R¹ and R² are independently H, C1-C20 organic groups that can be substituted or unsubstituted straight chain or branched hydrocarbyl group, substituted or unsubstituted cyclic hydrocarbyl group, substituted or unsubstituted heterocyclic group, substituted or unsubstituted aromatic group, substituted or unsubstituted heteraromatic group, a biobeneficial moiety, or a bioactive agent. Some exemplary organic groups are methyl, ethyl, propyl, isopropyl, butyl, 2-butyl, pentyl, 2-pentyl, 3-pentyl, 1-hexyl, 2-hexyl, 3-hexyl, cyclopentyl, cyclohexyl, phenyl, benzyl, phosphoryl choline, hydroxyl, and/or carboxylic acid.

In one embodiment, the copolymer described herein has a structure of

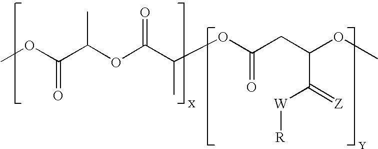

wherein X is a positive number ranging from about 0.01 to about 0.99,

wherein Y is a positive number ranging from about 0.99 to about 0,01,

wherein Z is O, S or NR¹,

wherein W is absence or O, S, or NR², and

where R, R¹ and R² are independently H, C1-C20 organic groups that can be substituted or unsubstituted straight chain or branched hydrocarbyl group, substituted or unsubstituted cyclic hydrocarbyl group, substituted or unsubstituted heterocyclic group, substituted or unsubstituted aromatic group, substituted or unsubstituted heteraromatic group, a biobeneficial moiety, or a bioactive agent. Some exemplary organic groups are methyl, ethyl, propyl, isopropyl, butyl, 2-butyl, pentyl, 2-pentyl, 3-pentyl, 1-hexyl, 2-hexyl, 3-hexyl, cyclopentyl, cyclohexyl, phenyl, benzyl, phosphoryl choline, hydroxyl, and/or carboxylic acid.

A. Moiety A Derived From Malolactonate or Malolactonic Acid

Malolactonates are esters of malolactonic acid. The structures of malolactonic acid and malolactonates are shown below in Scheme I:

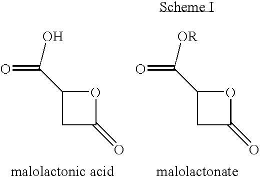

In Scheme I, the side group on malolactonate can be an organic group, e.g., a C1-C20 organic chemical group which can be substituted or unsubstituted straight chain or branched hydrocarbyl group, substituted or unsubstituted cyclic hydrocarbyl group, substituted or unsubstituted heterocyclic group, substituted or unsubstituted aromatic group, or substituted or unsubstituted heteraromatic group. Some exemplary organic groups are methyl, ethyl, propyl, isopropyl, butyl, 2-butyl, pentyl, 2-pentyl, 3-pentyl, 1-hexyl, 2-hexyl, 3-hexyl, cyclopentyl, cyclohexyl, phenyl, benzyl, phosphoryl choline, hydroxyl, and/or carboxylic acid.

In some embodiments, the malolactonate can be a compound having the following general structure (Scheme IA): 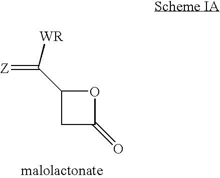
where Z is O, S or NR¹,
where W is absence or O, S, or NR²,

where R, R¹ and R² are independently H, C1-C20 organic chemical group which can be substituted or unsubstituted straight chain or branched hydrocarbyl group, substituted or unsubstituted cyclic hydrocarbyl group, substituted or unsubstituted heterocyclic group, substituted or unsubstituted aromatic group, or substituted or unsubstituted heteraromatic group. Some exemplary organic groups are methyl, ethyl, propyl, isopropyl, butyl, 2-butyl, pentyl, 2-pentyl, 3-pentyl, 1-hexyl, 2-hexyl, 3-hexyl, cyclopentyl, cyclohexyl, phenyl, benzyl, phosphoryl choline, hydroxyl, and/or carboxylic acid.

B. Synthesis of Malolactonate

The synthesis of malolactonic acid or malolactonates is well documented. For example, benzyl malolactonate can be synthesized via the two different routes shown in Scheme II:

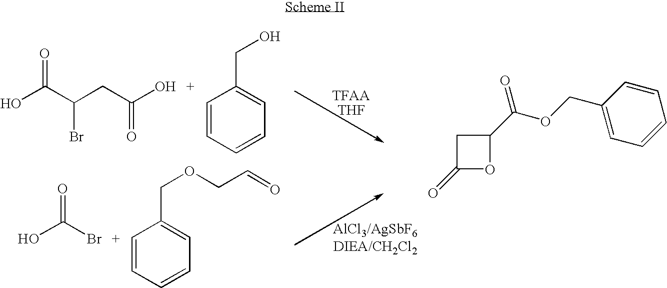

In the top route, benzyl alcohol can react with α-bromosuccinic acid in the presence of trifluoroacetic acid (TFAA) in a solvent such as tetrahydrofuran (THF) (He, B., et al., Biomaterials 25:5239 (2004). In the lower route, acetyl acyl bromide can react with benzyloxy aldehyde to form benzyl malolactonate. In this route, other groups such as protected amines, hydroxyl, or esters can be introduced as the malolactonate's side groups.

C Moiety B Derivedfrom Another Biocompatible Material

Moiety B can be derived from any biocompatible material capable of copolymerization with malolactonate or where the copolymer described herein is a block copolymer, capable of forming a block copolymer with a block containing moiety A. Where the copolymer described herein is a block copolymer, the material forming the moiety B block can be any biocompatible material such as biocompatible polymer. Some representative biocompatible polymers capable of forming the moiety B block includes, but are not limited to, poly(ester amide), polyhydroxyalkanoates (PHA), poly(3-hydroxyalkanoates) such as poly(3-hydroxypropanoate), poly(3-hydroxybutyrate), poly(3-hydroxyvalerate), poly(3-hydroxyhexanoate), poly(3-hydroxyheptanoate) and poly(3-hydroxyoctanoate), poly(4-hydroxyalkanaote) such as poly(4-hydroxybutyrate), poly(4-hydroxyvalerate), poly(4-hydroxyhexanote), poly(4-hydroxyheptanoate), poly(4-hydroxyoctanoate) and copolymers including any of the 3-hydroxyalkanoate or 4-hydroxyalkanoate monomers described herein or blends thereof, poly(D,L-lactic acid), poly(L-lactic acid), poly(glycolic acid), poly(D,L-lactic acid-co-glycolic acid), poly(L-lactic acid-co-glycolic acid), polycaprolactone, poly(lactic acid-co-caprolactone), poly(glycolic acid-co-caprolactone), poly(dioxanone), poly(ortho esters), poly(anhydrides), poly(tyrosine carbonates) and derivatives thereof, poly(tyrosine ester) and derivatives thereof, poly(imino carbonates), poly(glycolic acid-co-trimethylene carbonate), polyphosphoester, polyphosphoester urethane, poly(amino acids), polycyanoacrylates, poly(trimethylene carbonate), poly(iminocarbonate), polyurethanes, polyphosphazenes, silicones, polyesters, polyolefins, polyisobutylene and ethylene-alphaolefin copolymers, acrylic polymers and copolymers, vinyl halide polymers and copolymers, such as polyvinyl chloride, polyvinyl ethers, such as polyvinyl methyl ether, polyvinylidene halides, such as polyvinylidene chloride, polyacrylonitrile, polyvinyl ketones, polyvinyl aromatics, such as polystyrene, polyvinyl esters, such as polyvinyl acetate, copolymers of vinyl monomers with each other and olefins, such as ethylene-methyl methacrylate copolymers, acrylonitrile-styrene copolymers, ABS resins, and ethylene-vinyl acetate copolymers, polyamides, such as Nylon 66 and polycaprolactam, alkyd resins, polycarbonates, polyoxymethylenes, polyimides, polyethers, poly(glyceryl sebacate), poly(propylene fumarate), poly(n-butyl methacrylate), poly(sec-butyl methacrylate), poly(isobutyl methacrylate), poly(tert-butyl methacrylate), poly(n-propyl methacrylate), poly(isopropyl methacrylate), poly(ethyl methacrylate), poly(methyl methacrylate), epoxy resins, polyurethanes, rayon, rayon-triacetate, cellulose acetate, cellulose butyrate, cellulose acetate butyrate, cellophane, cellulose nitrate, cellulose propionate, cellulose ethers, carboxymethyl cellulose, polyethers such as poly(ethylene glycol) (PEG), copoly(ether-esters) (e.g. PEO/PLA), polyalkylene oxides such as poly(ethylene oxide), poly(propylene oxide), poly(ether ester), polyalkylene oxalates, polyphosphazenes, phosphoryl choline, choline, poly(aspirin), polymers and co-polymers of hydroxyl bearing monomers such as HEMA, hydroxypropyl methacrylate (HPMA), hydroxypropylmethacrylamide, PEG acrylate (PEGA), PEG methacrylate, 2-methacryloyloxyethylphosphorylcholine (MPC) and n-vinyl pyrrolidone (VP), carboxylic acid bearing monomers such as methacrylic acid (MA), acrylic acid (AA), alkoxymethacrylate, alkoxyacrylate, and 3-trimethylsilylpropyl methacrylate (TMSPMA), poly(styrene-isoprene-styrene)-PEG (SIS-PEG), polystyrene-PEG, polyisobutylene-PEG, polycaprolactone-PEG (PCL-PEG), poly(lactic acid-co-PEG) (PLA-PEG), poly(methyl methacrylate)-PEG (PMMA-PEG), polydimethylsiloxane-co-PEG (PDMS-PEG), poly(vinylidene fluoride)-PEG (PVDF-PEG), PLURONIC™ surfactants (polypropylene oxide-co-polyethylene glycol), poly(tetramethylene glycol), hydroxy functional poly(vinyl pyrrolidone), biomolecules such as collagen, chitosan, alginate, fibrin, fibrinogen, cellulose, starch, collagen, dextran, dextrin, fragments and derivatives of hyaluronic acid, heparin, fragments and derivatives of heparin, glycosamino glycan (GAG), GAG derivatives, polysaccharide, elastin, chitosan, alginate, and combinations thereof. In some embodiments, the polymer can exclude any one of the aforementioned polymers.

As used herein, the terms D,L-lactide, L-lactide, D,L-lactide-co-glycolide), and poly(L-lactide-co-glycolide) can be used interchangeably with the terms poly(D,L-lactic acid), poly(L-lactic acid), poly(D,L-lactic acid-co-glycolic acid), or poly(L-lactic acid-co-glycolic acid), respectively.

Where the polymer containing provided herein is a random copolymer, moiety B can be derived from monomers such as D,L-lactic acid, L-lactic acid, glycolic acid, glycolide, meso-lactide, racemic-D,L-lactide, lactone, caprolactone, trimethylene carbonate, dioxanone, hydroxybytyric acid, and/or hydroxyvaleric acid.

D. Method of Preparation

Malolactonates such as benzyl malolactonate can be polymerized with other lactones and/or lactides enzymatically or using a catalyst such as stanneous octoate. Polymeric materials with various attributes, e.g., materials with high molecular weights and narrow polydispersities, can be prepared. Scheme III shows an embodiment of the present invention, which shows copolymerization of benzyl malolactonate with lactide using stanneous octoate as a catalyst. The polymers prepared according to Scheme III are random copolymers.

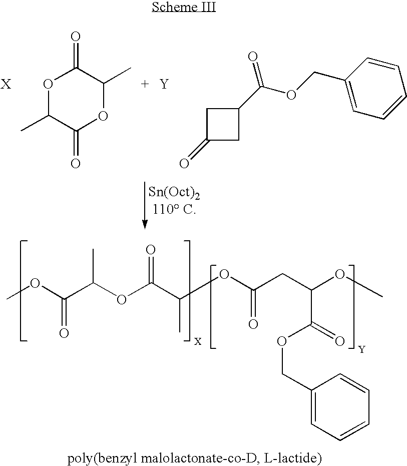

Where the polymer provided herein is a block copolymer containing at least one block derived from malolactonate or malolactic acid, the block copolymer can be prepared by coupling poly(malolactonate) or poly(malolactic acid) with a moiety derived from a biocompatible polymer described above.

Methods of forming copolymers are well established in the art (see, e.g., Polymer Synthesis: Theory and Practice. Braun, D., Cherdron, H., Rehahn, M., Ritter, H., Voit, B., 4th ed., Springer, 2005). An exemplary method of making the copolymer described herein is as follows.

In one embodiment, the initiator, hexanediol (Ig), DL-lactide (2.5 g) and the benzyl malolactonate are dissolved in anhydrous toluene. Three azeotropic distillations are performed from toluene under reduced atmosphere. The mixture is then added about 2 mL of anhydrous toluene under argon and then heated to about 110° C. Once the reagents are dissolved, about 12 mg of stanneous octoate is added and let react for 15 hours. The thus formed block copolymer can be dissolved in acetone and precipitated in cold methanol and then filtered out and dried under vacuum for 3 days at 60° C.

In some embodiments, the monomeric malolactonate bears a protective side group. The protective side group on malolactonate can be removed after polymerization. For example, the benzyl side groups on poly(benzyl malolactonate-co-D,L-lactide) prepared according to Scheme III can be removed by, for example, catalytic hydrogenation, to yield a carboxylic acid functionality on the polymer backbone (Scheme IV):

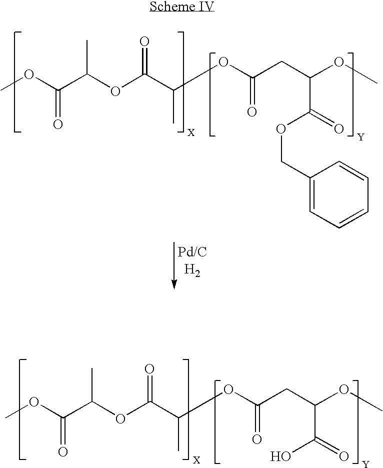

A side product of the hydrogenation reaction shown in Scheme IV is benzyl alcohol, which can be easily removed by known procedures such as solvent extraction or distillation.

Removable protective groups on side groups of malolactonate include, for example, heptyl ester (enzymatically cleavable), t-butyl ester, phenyl ester, trimethylsilyl ester (TMS), or t-butyldimethylsilyl ester (tBDMS). Some other protective groups can be found in Theodora W. Greene, Peter G. M. Wuts, “Protective groups in Organic Chemistry”, 3^{rd} Ed., Wiley, 1999.

E. Conjugation of Biobeneficial Moieties

As described above, the copolymer disclosed herein may contain an acidic group or a basic group. An acidic group such as carboxylic acid or a basic group such as an amino group can be used to tailor the degradation properties of the polymeric material since the degradation of lactides can be accelerated in an acidic environment.

The carboxylic acid group can also be used to attach moieties such as biobeneficial material and/or a drug(s) onto the polymer backbone. For example, poly(ethylene glycol) (PEG) with a hydroxyl terminal group can be coupled by esterification. Similarly, a drug or a peptide with a single unprotected hydroxyl can also be attached to this carboxyl acid group. Bioactive agents with other functionalities, e.g., amine groups, thiol groups, or carboxylic groups can also be attached to the polymer backbone via the carboxylic acid group or amino group on the polymer. Some illustrative methods of attaching a biobeneficial material or a bioactive agent (drug) onto a polymer via carboxylic acid group are described in U.S. application Ser, Nos. 10/871,658 and 10/857,141. A biobeneficial material is one which enhances the biocompatibility of a device by being non-fouling, hemocompatible, actively non-thrombogenic, or anti-inflammatory, all without depending on the release of a pharmaceutically active agent.

The biobeneficial materials that can be attached to the copolymer described-herein include, but are not limited to, non-fouling moieties such as PEG, phosphoryl choline and poly(vinyl pyrrolidinone) and other biobeneficial materials such as heparin, heparin fragments, heparin derivatives, hyaluronic acid, laminin, osteopontin, A, B- and C-natriuretic peptide, and/or CD-34 antibody.

One or more bioactive agents may also be attached to the copolymer described herein.

### Other Biocompatible Polymers

In some embodiments, the polymer described herein can form a device (e.g., absorbable stent) or a coating optionally with one or more other biocompatible polymers. The combination can be mixed, blended, or coated in separate layers. The additional biocompatible polymer can be biodegradable (both bioerodable or bioabsorbable) or nondegradable, and can be hydrophilic or hydrophobic. Hydrophilic is defined to have a δ value greater than about 8.5 cm³/mole, e.g., a δ value of about 8.5 cm³/mole, about 9.5 cm³/mole, about 10.5 cm³/mole or about 11.5 cm³/mole. δ Value is a hydrophobicity scale commonly used in the art of polymer or protein materials, which is determined by the following equation:
δ=(ΔE/V)^{1/2}
where ΔE is the energy of vaporization, cal/mole, and V is the molar volume, cm³/mole.

Representative biocompatible polymers include, but are not limited to, poly(ester amide), polyhydroxyalkanoates (PHA), poly(3-hydroxyalkanoates) such as poly(3-hydroxypropanoate), poly(3-hydroxybutyrate), poly(3-hydroxyvalerate), poly(3-hydroxyhexanoate), poly(3-hydroxyheptanoate) and poly(3-hydroxyoctanoate), poly(4-hydroxyalkanaote) such as poly(4-hydroxybutyrate), poly(4-hydroxyvalerate), poly(4-hydroxyhexanote), poly(4-hydroxyheptanoate), poly(4-hydroxyoctanoate) and copolymers including any of the 3-hydroxyalkanoate or 4-hydroxyalkanoate monomers described herein or blends thereof, poly(D,L-lactide), poly(L-lactide), polyglycolide, poly(D,L-lactide-co-glycolide), poly(L-lactide-co-glycolide), polycaprolactone, poly(lactide-co-caprolactone), poly(glycolide-co-caprolactone), poly(dioxanone), poly(ortho esters), poly(anhydrides), poly(tyrosine carbonates) and derivatives thereof, poly(tyrosine ester) and derivatives thereof, poly(imino carbonates), poly(glycolic acid-co-trimethylene carbonate), polyphosphoester, polyphosphoester urethane, poly(amino acids), polycyanoacrylates, poly(trimethylene carbonate), poly(iminocarbonate), polyurethanes, polyphosphazenes, silicones, polyesters, polyolefins, polyisobutylene and ethylene-alphaolefin copolymers, acrylic polymers and copolymers, vinyl halide polymers and copolymers, such as polyvinyl chloride, polyvinyl ethers, such as polyvinyl methyl ether, polyvinylidene halides, such as polyvinylidene chloride, polyacrylonitrile, polyvinyl ketones, polyvinyl aromatics, such as polystyrene, polyvinyl esters, such as polyvinyl acetate, copolymers of vinyl monomers with each other and olefins, such as ethylene-methyl methacrylate copolymers, acrylonitrile-styrene copolymers, ABS resins, and ethylene-vinyl acetate copolymers, polyamides, such as Nylon 66 and polycaprolactam, alkyd resins, polycarbonates, polyoxymethylenes, polyimides, polyethers, poly(glyceryl sebacate), poly(propylene fumarate), poly(n-butyl methacrylate), poly(sec-butyl methacrylate), poly(isobutyl methacrylate), poly(tert-butyl methacrylate), poly(n-propyl methacrylate), poly(isopropyl methacrylate), poly(ethyl methacrylate), poly(methyl methacrylate), epoxy resins, polyurethanes, rayon, rayon-triacetate, cellulose acetate, cellulose butyrate, cellulose acetate butyrate, cellophane, cellulose nitrate, cellulose propionate, cellulose ethers, carboxymethyl cellulose, polyethers such as poly(ethylene glycol) (PEG), copoly(ether-esters) (e.g. PEO/PLA), polyalkylene oxides such as poly(ethylene oxide), poly(propylene oxide), poly(ether ester), polyalkylene oxalates, polyphosphazenes, phosphoryl choline, choline, poly(aspirin), polymers and co-polymers of hydroxyl bearing monomers such as HEMA, hydroxypropyl methacrylate (HPMA), hydroxypropylmethacrylamide, PEG acrylate (PEGA), PEG methacrylate, 2-methacryloyloxyeth.ylphosphorylcholine (MPC) and n-vinyl pyrrolidone (VP), carboxylic acid bearing monomers such as methacrylic acid (MA), acrylic acid (AA), alkoxymethacrylate, alkoxyacrylate, and 3-trimethylsilylpropyl methacrylate (TMSPMA), poly(styrene-isoprene-styrene)-PEG (SIS-PEG), polystyrene-PEG, polyisobutylene-PEG, polycaprolactone-PEG (PCL-PEG), PLA-PEG, poly(methyl methacrylate)-PEG (PMMA-PEG), polydimethylsiloxane-co-PEG (PDMS-PEG), poly(vinylidene fluoride)-PEG (PVDF-PEG), PLURONIC™ surfactants (polypropylene oxide-co-polyethylene glycol), poly(tetramethylene glycol), hydroxy functional poly(vinyl pyrrolidone), biomolecules such as collagen, chitosan, alginate, fibrin, fibrinogen, cellulose, starch, collagen, dextran, dextrin, fragments and derivatives of hyaluronic acid, heparin, fragments and derivatives of heparin, glycosamino glycan (GAG), GAG derivatives, polysaccharide, elastin, chitosan, alginate, or combinations thereof. In some embodiments, the copolymer described herein can exclude any one of the aforementioned polymers.

As used herein, the terms poly(D,L-lactide), poly(L-lactide), poly(D,L-lactide-co-glycolide), and poly(L-lactide-co-glycolide) can be used interchangeably with the terms poly(D,L-lactic acid), poly(L-lactic acid), poly(D,L-lactic acid-co-glycolic acid), or poly(L-lactic acid-co-glycolic acid), respectively.

### Biobeneficial Material

In some embodiments, the polymer described herein, with or without conjugation to biobeneficial moeties and/or bioactive agents as described herein, can form a device (e.g., absorbable stent) or a coating optionally with a biobeneficial material. The combination can be mixed, blended, or coated in separate layers. The biobeneficial material useful in the coatings described herein can be a polymeric material or non-polymeric material. The biobeneficial material is preferably non-toxic, non-antigenic and non-immunogenic. A biobeneficial material is one which enhances the biocompatibility of a device by being non-fouling, hemocompatible, actively non-thrombogenic, or anti-inflammatory, all without depending on the release of a pharmaceutically active agent.

Representative biobeneficial materials include, but are not limited to, polyethers such as poly(ethylene glycol), copoly(ether-esters) (e.g. PEO/PLA), polyalkylene oxides such as poly(ethylene oxide), poly(propylene oxide), poly(ether ester), polyalkylene oxalates, polyphosphazenes, phosphoryl choline, choline, poly(aspirin), polymers and co-polymers of hydroxyl bearing monomers such as hydroxyethyl methacrylate (HEMA), hydroxypropyl methacrylate (HPMA), hydroxypropylmethacrylamide, poly (ethylene glycol) acrylate (PEGA), PEG methacrylate, 2-methacryloyloxyethylphosphorylcholine (MPC) and n-vinyl pyrrolidone (VP), carboxylic acid bearing monomers such as methacrylic acid (MA), acrylic acid (AA), alkoxymethacrylate, alkoxyacrylate, and 3-trimethylsilylpropyl methacrylate (TMSPMA), poly(styrene-isoprene-styrene)-PEG (SIS-PEG), polystyrene-PEG, polyisobutylene-PEG, polycaprolactone-PEG (PCL-PEG), PLA-PEG, poly(methyl methacrylate)-PEG (PMMA-PEG), polydimethylsiloxane-co-PEG (PDMS-PEG), poly(vinylidene fluoride)-PEG (PVDF-PEG), PLURONIC™ surfactants (polypropylene oxide-co-polyethylene glycol), poly(tetramethylene glycol), hydroxy functional poly(vinyl pyrrolidone), biomolecules such as fibrin, fibrinogen, cellulose, starch, collagen, dextran, dextrin, hyaluronic acid, fragments and derivatives of hyaluronic acid, heparin, fragments and derivatives of heparin, glycosamino glycan (GAG), GAG derivatives, polysaccharide, elastin, chitosan, alginate, silicones, PolyActive™, and combinations thereof. In some embodiments, the coating can exclude any one of the aforementioned polymers.

The term PolyActive™ refers to a block copolymer having flexible poly(ethylene glycol) and poly(butylene terephthalate) blocks (PEGT/PBT). PolyActive™ is intended to include AB, ABA, BAB copolymers having such segments of PEG and PBT (e.g., poly(ethylene glycol)-block-poly(butyleneterephthalate)-block poly(ethylene glycol) (PEG-PBT-PEG).

In a preferred embodiment, the biobeneficial material can be a polyether such as poly (ethylene glycol) (PEG) or polyalkylene oxide.

### Bioactive Agents

In some embodiments, the polymer described herein, with or without conjugation to biobeneficial moieties and/or bioactive agents as described herein, can form a device (e.g., absorbable stent) or a coating optionally with one or more bioactive agents. These bioactive agents can be any agent which is a therapeutic, prophylactic, or diagnostic agent. These agents can have anti-proliferative or anti-inflammmatory properties or can have other properties such as antineoplastic, antiplatelet, anti-coagulant, anti-fibrin, antithrombonic, antimitotic, antibiotic, antiallergic, antioxidant as well as cystostatic agents, agents that promote the healing of the endothelium such as NO releasing or generating agents, agents that attract endothelial progenitor cells, or agents that promote the attachment, migration and proliferation of endothelial cells (CNP, cRGD) while quenching smooth muscle cell proliferation. Examples of suitable therapeutic and prophylactic agents include synthetic inorganic and organic compounds, proteins and peptides, polysaccharides and other sugars, lipids, and DNA and RNA nucleic acid sequences having therapeutic, prophylactic or diagnostic activities. Nucleic acid sequences include genes, antisense molecules which bind to complementary DNA to inhibit transcription, and ribozymes. Some other examples of other bioactive agents include antibodies, receptor ligands, enzymes, adhesion peptides, blood clotting factors, inhibitors or clot dissolving agents such as streptokinase and tissue plasminogen activator, antigens for immunization, hormones and growth factors, oligonucleotides such as antisense oligonucleotides and ribozymes and retroviral vectors for use in gene therapy. Examples of anti-proliferative agents include rapamycin and its functional or structural derivatives, 40-O-2-hydroxy)ethyl-rapamycin (everolimus), and its functional or structural derivatives, paclitaxel and its functional and structural derivatives. Examples of rapamycin derivatives include methyl rapamycin (ABT-578), 40-O-(3-hydroxy)propyl-rapamycin, 40-O-[2-(2-hydroxy)ethoxy]ethyl-rapamycin, and 40-O-tetrazole-rapamycin. Examples of paclitaxel derivatives include docetaxel. Examples of antineoplastics and/or antimitotics include methotrexate, azathioprine, vincristine, vinblastine, fluorouracil, doxorubicin hydrochloride (e.g. Adriamycin® from Pharmacia & Upjohn, Peapack N.J.), and mitomycin (e.g. Mutamycin® from Bristol-Myers Squibb Co., Stamford, Conn.). Examples of such antiplatelets, anticoagulants, antifibrin, and antithrombins include sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIIb/IIIa platelet membrane receptor antagonist antibody, recombinant hirudin, thrombin inhibitors such as Angiomax a (Biogen, Inc., Cambridge, Mass.), calcium channel blockers (such as nifedipine), colchicine, fibroblast growth factor (FGF) antagonists, fish oil (omega 3-fatty acid), histamine antagonists, lovastatin (an inhibitor of HMG-CoA reductase, a cholesterol lowering drug, brand name Mevacor® from Merck & Co., Inc., Whitehouse Station, N.J.), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), nitric oxide or nitric oxide donors, super oxide dismutases, super oxide dismutase mimetic, 4-amino-2,2,6,6-tetramethylpiperidine-1-oxyl (4-amino-TEMPO), estradiol, anticancer agents, dietary supplements such as various vitamins, and a combination thereof. Examples of anti-inflammatory agents including steroidal and non-steroidal anti-inflammatory agents include tacrolimus, dexamethasone, clobetasol, combinations thereof. Examples of such cytostatic substance include angiopeptin, angiotensin converting enzyme inhibitors such as captopril (e.g. Capoten® and Capozide® from Bristol-Myers Squibb Co., Stamford, Conn.), cilazapril or lisinopril (e.g. Prinivil® and Prinzide® from Merck & Co., Inc., Whitehouse Station, N.J.). An example of an antiallergic agent is permirolast potassium. Other therapeutic substances or agents which may be appropriate include alpha-interferon, pimecrolimus, imatinib mesylate, midostaurin, bioactive RGD, and genetically engineered epithelial cells. The foregoing substances can also be used in the form of prodrugs or co-drugs thereof. The foregoing substances also include metabolites thereof and/or prodrugs of the metabolites. The foregoing substances are listed by way of example and are not meant to be limiting. Other active agents which are currently available or that may be developed in the future are equally applicable.

The dosage or concentration of the bioactive agent required to produce a favorable therapeutic effect should be less than the level at which the bioactive agent produces toxic effects and greater than the level at which non-therapeutic results are obtained. The dosage or concentration of the bioactive agent can depend upon factors such as the particular circumstances of the patient, the nature of the trauma, the nature of the therapy desired, the time over which the ingredient administered resides at the vascular site, and if other active agents are employed, the nature and type of the substance or combination of substances. Therapeutic effective dosages can be determined empirically, for example by infusing vessels from suitable animal model systems and using immunohistochemical, fluorescent or electron microscopy methods to detect the agent and its effects, or by conducting suitable in vitro studies. Standard pharmacological test procedures to determine dosages are understood by one of ordinary skill in the art.

### Examples of Implantable Device

As used herein, an implantable device may be any suitable medical substrate that can be implanted in a human or veterinary patient. Examples of such implantable devices include self-expandable stents, balloon-expandable stents, stent-grafts, grafts (e.g., aortic grafts), artificial heart valves, cerebrospinal fluid shunts, pacemaker electrodes, catheters, and endocardial leads (e.g., FINELINE and ENDOTAK, available from Guidant Corporation, Santa Clara, Calif.). The underlying structure of the device can be of virtually any design. The device can be made of a metallic material or an alloy such as, but not limited to, cobalt chromium alloy (ELGILOY), stainless steel (316L), high nitrogen stainless steel, e.g., BIODUR 108, cobalt chrome alloy L-605, “MP35N,” “MP20N,” ELASTINITE (Nitinol), tantalum, nickel-titanium alloy, platinum-iridium alloy, gold, magnesium, or combinations thereof. “MP35N” and “MP20N” are trade names for alloys of cobalt, nickel, chromium and molybdenum available from Standard Press Steel Co., Jenkintown, Pa. “MP35N” consists of 35% cobalt, 35% nickel, 20% chromium, and 10% molybdenum. “MP20N” consists of 50% cobalt, 20% nickel, 20% chromium, and 10% molybdenum. Devices made from bioabsorbable or biostable polymers could also be used with the embodiments of the present invention. The device itself, such as a stent, can also be made from the described inventive polymers or polymer blends.

### Method of Use

In accordance with embodiments of the invention, a coating can be formed on an implantable device or prosthesis, e.g., a stent. For coatings including one or more active agents, the agent will retain on the medical device such as a stent during delivery and expansion of the device, and released at a desired rate and for a predetermined duration of time at the site of implantation.

Preferably, the medical device is a stent. The stent described herein is useful for a variety of medical procedures, including, by way of example, treatment of obstructions caused by tumors in bile ducts, esophagus, trachea/bronchi and other biological passageways. A stent having the above-described coating is particularly useful for treating occluded regions of blood vessels caused by abnormal or inappropriate migration and proliferation of smooth muscle cells, thrombosis, and restenosis. Stents may be placed in a wide array of blood vessels, both arteries and veins. Representative examples of sites include the iliac, renal, and coronary arteries.

For implantation of a stent, an angiogram is first performed to determine the appropriate positioning for stent therapy. An angiogram is typically accomplished by injecting a radiopaque contrasting agent through a catheter inserted into an artery or vein as an x-ray is taken. A guidewire is then advanced through the lesion or proposed site of treatment. Over the guidewire is passed a delivery catheter which allows a stent in its collapsed configuration to be inserted into the passageway. The delivery catheter is inserted either percutaneously or by surgery into the femoral artery, brachial artery, femoral vein, or brachial vein, and advanced into the appropriate blood vessel by steering the catheter through the vascular system under fluoroscopic guidance. A stent having the above-described coating may then be expanded at the desired area of treatment. A post-insertion angiogram may also be utilized to confirm appropriate positioning.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

What is claimed is:

1. A copolymer comprising units derived from malolactonate or malolactic acid.

2. The copolymer of claim 1 comprising a polymalolactonate block and a block selected from the group consisting of poly(ester amide), polyhydroxyalkanoates (PHA), poly(3-hydroxyalkanoates), poly(3-hydroxypropanoate), poly(3-hydroxybutyrate), poly(3-hydroxyvalerate), poly(3-hydroxyhexanoate), poly(3-hydroxyheptanoate), poly(3-hydroxyoctanoate), poly(4-hydroxyalkanaote), poly(4-hydroxybutyrate), poly(4-hydroxyvalerate), poly(4-hydroxyhexanote), poly(4-hydroxyheptanoate), poly(4-hydroxyoctanoate), copolymers including any of the 3-hydroxyalkanoate, 4-hydroxyalkanoate monomers or combinations thereof, poly(D,L-lactic acid), poly(L-lactic acid), poly(glycolic acid), poly(D,L-lactic acid-co-glycolic acid), poly(L-lactic acid-co-glycolic acid), polycaprolactone, poly(lactic acid-co-caprolactone), poly(glycolic acid-co-caprolactone), poly(dioxanone), poly(ortho esters), poly(anhydrides), poly(tyrosine carbonates), poly(tyrosine ester), poly(imino carbonates), poly(glycolic acid-co-trimethylene carbonate), polyphosphoester, polyphosphoester urethane, poly(amino acids), polycyanoacrylates, poly(trimethylene carbonate), poly(iminocarbonate), polyurethanes, polyphosphazenes, silicones, polyesters, polyolefins, polyisobutylene, ethylene-alphaolefin copolymers, acrylic polymers and copolymers, vinyl halide polymers and copolymers, polyvinyl chloride, polyvinyl ethers, polyvinyl methyl ether, polyvinylidene halides, polyvinylidene chloride, polyacrylonitrile, polyvinyl ketones, polyvinyl aromatics, polystyrene, polyvinyl esters, polyvinyl acetate, copolymers of vinyl monomers with each other and olefins, ethylene-methyl methacrylate copolymers, acrylonitrile-styrene copolymers, ABS resins, ethylene-vinyl acetate copolymers, polyamides, Nylon 66 and polycaprolactam, alkyd resins, polycarbonates, polyoxymethylenes, polyimides, polyethers, poly(glyceryl sebacate), poly(propylene fumarate), poly(n-butyl methacrylate), poly(sec-butyl methacrylate), poly(isobutyl methacrylate), poly(tert-butyl methacrylate), poly(n-propyl methacrylate), poly(isopropyl methacrylate), poly(ethyl methacrylate), poly(methyl methacrylate), epoxy resins, polyurethanes, rayon, rayon-triacetate, cellulose acetate, cellulose butyrate, cellulose acetate butyrate, cellophane, cellulose nitrate, cellulose propionate, cellulose ethers, carboxymethyl cellulose, polyethers such as poly(ethylene glycol) (PEG), copoly(ether-esters), poly(ethylene oxide-co-lactic acid) (PEO/PLA), polyalkylene oxides, poly(ethylene oxide), poly(propylene oxide), poly(ether ester), polyalkylene oxalates, polyphosphazenes, phosphoryl choline, choline, poly(aspirin), polymers and co-polymers of HEMA, hydroxypropyl methacrylate (HPMA), hydroxypropylmethacrylamide, PEG acrylate (PEGA), PEG methacrylate, 2-methacryloyloxyethylphosphorylcholine (MPC) and n-vinyl pyrrolidone (VP), methacrylic acid,(MA), acrylic acid (AA), alkoxymethacrylate, alkoxyacrylate, and 3-trimethylsilylpropyl methacrylate (TMSPMA), poly(styrene-isoprene-styrene)-PEG (SIS-PEG), polystyrene-PEG, polyisobutylene-PEG, polycaprolactone-PEG (PCL-PEG), poly(lactic acid-co-PEG) (PLA-PEG), poly(methyl methacrylate)-PEG (PMMA-PEG), polydimethylsiloxane-co-PEG (PDMS-PEG), poly(vinylidene fluoride)-PEG (PVDF-PEG), PLURONIC™ surfactants, poly(tetramethylene glycol), hydroxy fuinctional poly(vinyl pyrrolidone), biomolecules, collagen, chitosan, alginate, fibrin, fibrinogen, cellulose, starch, collagen, dextran, dextrin, hyaluronic acid, fragments of hyaluronic acid, heparin, fragments of heparin, glycosamino glycan (GAG) , polysaccharide, elastin, chitosan, alginate, derivatives thereof, and combinations thereof.

3. The copolymer of claim 1, further comprising repeating units derived from lactic acid.

4. The copolymer of claim 1 comprising repeating units of the following structure: 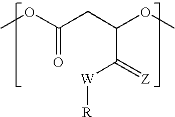
wherein Z is O, S or NR¹,
wherein W is absence or O, S, or NR², and
where R, R¹ and R² are independently H, C1-C20 substituted or unsubstituted straight chain or branched hydrocarbyl group, substituted or unsubstituted cyclic hydrocarbyl group, substituted or unsubstituted heterocyclic group, substituted or unsubstituted aromatic group, substituted or unsubstituted heteraromatic group, a biobeneficial moiety, or a bioactive agent.

5. The copolymer of claim 4,
wherein W is absence, O, or NH,
wherein R is H, benzyl, a biobeneficial moiety, or a bioactive agent.

6. The copolymer of claim 1 having the structure of 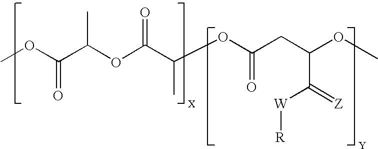
wherein X is a positive number ranging from about 0.01 to about 0.99,
wherein Y is a positive number ranging from about 0.99 to about 0,01,
wherein Z is O, S or NR¹,
wherein W is absence or O, S, or NR², and
where R, R¹ and R² are independently H, C1-C20 substituted or unsubstituted straight chain or branched hydrocarbyl group, substituted or unsubstituted cyclic hydrocarbyl group, substituted or unsubstituted heterocyclic group, substituted or unsubstituted aromatic group, substituted or unsubstituted heteraromatic group, a biobeneficial moiety, or a bioactive agent.

7. The copolymer of claim 6, which is a random copolymer.

8. The copolymer of claim 1, further comprising a biobeneficial moiety attached thereto.

9. The copolymer of claim 8, wherein the biobeneficial moiety is selected from the group consisting of PEG, phosphoryl choline and poly(vinyl pyrrolidinone), heparin, heparin fragments, heparin derivatives, hyaluronic acid, laminin, osteopontin, A, B- and C-natriuretic peptide, CD-34 antibody, and combinations thereof.

10. The copolymer of claim 1, further comprising a bioactive agent attached thereto.

11. The copolymer of claim 10, wherein the bioactive agent is a peptide or a drug.

12. An implantable device comprising a coating formed of a material that comprises the copolymer of claim 1.

13. An implantable device comprising a coating formed of a material that comprises the copolymer of claim 4.

14. An implantable device comprising a coating formed of a material that comprises the copolymer of claim 5.

15. An implantable device comprising a coating formed of a material that comprises the copolymer of claim 6.

16. An implantable device comprising a coating formed of a material that comprises the copolymer of claim 8.

17. An implantable device comprising a coating formed of a material that comprises the copolymer of claim 9.

18. An implantable device comprising a coating formed of a material that comprises the copolymer of claim 10.

19. An implantable device comprising a coating formed of a material that comprises the copolymer of claim 11.

20. The implantable device of claim 12, further comprising a bioactive agent.

21. The implantable device of claim 13, further comprising a bioactive agent.

22. The implantable device of claim 14, further comprising a bioactive agent.

23. The implantable device of claim 15, further comprising a bioactive agent.

24. The implantable device of claim 22, wherein the bioactive agent is selected from the group consisting of paclitaxel, docetaxel, estradiol, nitric oxide donors, super oxide dismutases, super oxide dismutases mimics, 4-amino-2,2,6,6-tetramethylpiperidine-1-oxyl (4-amino-TEMPO), tacrolimus, dexamethasone, rapamycin, rapamycin derivatives, 40-O-(2-hydroxy)ethyl-rapamycin (everolimus), 40-O-(3-hydroxy)propyl-rapamycin, 40-O-[2-(2-hydroxy)ethoxy]ethyl-rapamycin, and 40-O-tetrazole-rapamycin, 40-epi-(N1-tetrazolyl)-rapamycin (ABT-578), clobetasol, pimecrolimus, imatinib mesylate, midostaurin, prodrugs thereof, co-drugs thereof, and a combination thereof.

25. The implantable device of claim 20 which is a stent.

26. The implantable device of claim 24, which is a stent.

27. An absorbable stent formed of a material comprising the copolymer of claim 6.

28. A method of treating a disorder in a patient comprising implanting in the patient the impantable device of claim 19, wherein the disorder is selected from the group consisting of atherosclerosis, thrombosis, restenosis, hemorrhage, vascular dissection or perforation, vascular aneurysm, vulnerable plaque, chronic total occlusion, patent foramen ovale, claudication, anastomotic proliferation for vein and artificial grafts, bile duct obstruction, ureter obstruction, tumor obstruction, and combinations thereof
